(19) United States
(12) Patent Application Publication
(10) Pub. No.: US 2009/0214464 A1
(43) Pub. Date: Aug. 27, 2009

(54) COMPOUNDS FOR FLAVIVIRIDAE TREATMENT

(76) Inventor: Beat Weidmann, Winchester, MA (US)

Correspondence Address: MCCARTER & ENGLISH, LLP WILMINGTON;BASIL S. KRIKELIS, Renaissance Centre, 405 N. King Street, 8th Floor, WILMINGTON, DE, 19801, US

(21) Appl. No.: 11/719,684

(22) PCT Filed: Dec. 20, 2005
(86) PCT No.: PCT/US2005/046058

§ 371 Date: Jan. 29, 2009

Related U.S. Application Data
(60) Provisional application No. 60/638,676, filed on Dec. 23, 2004.

(51) Int. Cl. A61K 38/13; A61P 31/12; A61K 38/20; A61K 38/21

(52) U.S. Cl. 424/85.2; 514/11; 424/85.4

(57) ABSTRACT

Disclosed are non-immunosuppressive cyclophilin-binding cyclosporins, e.g. of formula I, Ia or II as defined herein, having useful properties in the prevention or treatment of Flaviviridae infections and Flaviviridae induced disorders.

### FIELD OF THE INVENTION

The present invention relates to a new use for non-immunosuppressive cyclosporins in the treatment of Flaviviridae viral infections and induced disorders.

### BACKGROUND OF THE INVENTION

The cyclosporins comprise a class of structurally distinctive, cyclic, poly-N-methylated undecapeptides, commonly possessing pharmacological, in particular immunosuppressive, or anti-inflammatory activity. The first of the cyclosporins to be isolated was the naturally occurring fungal metabolite Ciclosporin or Cyclosporine, also known as cyclosporin A.

It is well established that cyclosporin A acts by interfering with the process of T cell activation by blocking transcription initiation of IL-2. Cyclosporin A has been shown to form a complex with a 17 kD cytosolic protein named as cyclophilin, that occurs in many cell types and has been shown to be identical to peptidyl-prolyl cis-trans isomerase, an enzyme involved in protein folding.

However, it was found that binding to cyclophilin is a necessary but not a sufficient criterion for immunosuppressive activity. The cyclosporin A/cyclophilin complex can also associate with the cellular protein named calcineurin (CN) which belongs to the phosphatase superfamily. This binding abrogates its phosphatase activity, resulting in silencing of transcription factor NF-AT. The inhibition of the CN/NF-AT pathway is the essential mechanism for cyclosporin A mediated immunosuppression.

Cyclosporins which bind strongly to cyclophilin but are not immunosuppressive have been identified. A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Peris, Eds., Academic Press, N.Y. pp. 227-239 (1979). Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway.

EP 0 484 281 A1 and U.S. Pat. No. 5,767,069 disclose the use of non-immunosuppressive cyclosporins in the treatment of AIDS or AIDS-related disorders. As disclosed in application EP 2004/009804, non-immunosuppressive cyclosporins which bind to cyclophilin have also been found to have an inhibitory effect on Hepatitis C virus (HCV).

Persistent infection by HCV, which has been identified as the major causative agent of non-A, non-B hepatitis has been considered closely related to liver diseases such as chronic hepatitis, liver cirrhosis or hepatocellular carcinoma. The development of these liver diseases is a major public heath problem. Effective anti-HCV therapy is restricted to therapy with interferon or a combination of interferon and ribavirin. However, since the virus is not eliminated from about a half of the HCV patients treated with these known agents, there is still a strong need for alternative anti-HCV agents.

Hepatitis C infections or HCV induced disorders are e.g. chronic hepatitis, liver cirrhosis or liver cancer, e.g. hepatocellular carcinoma. The non-immunosuppressive cyclophilin-binding cyclosporins may also be used for example as a prophylactic treatment of neonates born to HCV infected mothers or of healthcare workers exposed to the virus, or of transplant recipients, e.g. organ or tissue transplant recipients, e.g. liver transplant, to eliminate possible recurrent HCV infection after transplantation.

HCV is the sole member of the genus hepaciviruses. Hepaciviruses, flaviviruses and pestiviruses are three distinct genera of the Flaviviridae family of viruses. The flavivirus genus includes more than 68 members separated into groups on the basis of serological relatedness (Calisher et al., J. Gen. Virol. 1993, 70:37-43). Clinical symptoms vary and include fever, encephalitis and hemorrhagic fever (Fields Virology, Eds: Fields, B. N., Knipe, D. M., and Howley, P. M., Lippincott Raven publishers, Philadelphia, Pa., 1996, Chapter 31, 931-959). Flaviviruses of global concern that are associated with human disease include the dengue hemorrhagic fever viruses (DHF), yellow fever virus, shock syndrome and Japanese encephalitis virus (Halstead, S. B., Rev. Infect. Dis., 1984, 6, 251-264; Halstead, S. B., Science, 239:476-481, 1988; Monath, T. P., New Eng. J. Med. 1988, 319, 641-643).

The pestivirus genus includes bovine viral diarrhea virus (BVDV), classical swine fever virus (CSFV, also called hog cholera virus) and border disease virus (BDV) of sheep (Moennig, V. et al., Adv. Vir. Res. 1992, 41, 53-98). Pestivirus infections of domesticated livestock (cattle, pigs, and sheep) cause significant economic losses worldwide. BVD causes mucosal disease in cattle and is of significant economic importance to the livestock industry (Meyers, G. and Thiel, H.-J., Advances in Virus Research, 1996, 47, 53-118; Moening V., t al. Adv. Vir. Res. 1992, 41, 53-98). Human pestiviruses have not been as extensively characterized as the animal pestiviruses. However, serological surveys indicate considerable pestivirus exposure in humans.

Pestiviruses and hepaciviruses are closely related virus groups within the Flaviviridae family. Other closely related viruses in this family include the GB virus A, GB virus A-like agents, GB virus-B and GB virus-C (also called hepatitis G virus, HGV). The hepacivirus group (hepatitis C virus; HCV) consists of a number of closely related but genotypically distinguishable viruses that infect humans. There are approximately 6 HCV genotypes and more than 50 subtypes. Due to the similarities between pestiviruses and hepaciviruses, combined with the poor ability of hepaciviruses to grow efficiently in cell culture, bovine viral diarrhea virus (BVD) is often used as a surrogate to study the HCV virus.

The genetic organization of pestiviruses and hepaciviruses is very similar. These positive stranded RNA viruses possess a single large open reading frame (ORF) encoding all the viral proteins necessary for virus replication. These proteins are expressed as a polyprotein that is co- and post-translationally processed by both cellular and virus-encoded proteinases to yield the mature viral proteins. The viral proteins responsible for the replication of the viral genome RNA are located within approximately the carboxy-terminal. Two-thirds of the ORF are termed nonstructural (NS) proteins. The genetic organization and polyprotein processing of the nonstructural protein portion of the ORF for pestiviruses and hepaciviruses is very similar. For both the pestiviruses and hepaciviruses, the mature nonstructural (NS) proteins, in sequential order from the amino-terminus of the nonstructural protein coding region to the carboxy-terminus of the ORF, consist of p7, NS2, NS3, NS4A, NS4B, NS5A, and NS5B. The genetic organization of HCV is depicted in FIG. 1.

The NS proteins of pestiviruses and hepaciviruses share sequence domains that are characteristic of specific protein functions. For example, the NS3 proteins of viruses in both groups possess amino acid sequence motifs characteristic of serine proteinases and of helicases (Gorbalenya et al. (1988) Nature 333:22; Bazan and Fletterick (1989) Virology 171:637-639; Gorbalenya et al. (1989) Nucleic Acid Res. 17:3889-3897). Similarly, the NS5B proteins of pestiviruses and hepaciviruses have the motifs characteristic of RNA-directed RNA polymerases (Koonin, E. V. and Dolja, V. V. (1993) Crit. Rev. Biochem. Molec. Biol. 28:375-430).

The actual roles and functions of the NS proteins of pestiviruses and hepaciviruses in the lifecycle of the viruses are directly analogous. In both cases, the NS3 serine proteinase is responsible for all proteolytic processing of polyprotein precursors downstream of its position in the ORF (Wiskerchen and Collett (1991) Virology 184:341-350; Bartenschlager et al. (1993) J. Virol. 67:3835-3844; Eckart et al. (1993) Biochem. Biophys. Res. Comm. 192:399-4067; Grakoui et al. (1993) J. Virol. 67:2832-2843; Grakoui et al. (1993) Proc. Natl. Aced. Sci. USA 90:10583-10587; Hijikata et al. (1993) J. Virol. 67:4665-4675; Tome et al. (1993) J. Virol. 71:5312-5322). The NS3 proteins of both viruses also function as a helicase (Kim et al. (1995) Biochem. Biophys. Res. Comm. 215:160-166; Jin and Peterson (1995) Arch. Biochem. Biophys., 323:47-53; Warrener and Collett (1995) J. Virol. 69:1720-1726). Finally, the NS5B proteins of pestiviruses and hepaciviruses have the predicted RNA-directed RNA polymerase activity (Behrens et al. (1996) EMBO J. 15:12-22; Lechmann et al. (1977) J. Virol. 71:8416-8428; Yuan et al. (1997) Biochem. Biophys. Res. Comm. 232:231-235; Hagedorn, PCT WO 97/12033; Zhong et al. (1998) J. Virol. 72:9365-9369).

The present invention provides the use of non-immunosuppressive cyclophilin-binding cyclosporins in the prevention or treatment of Flaviviridae virus infections and induced disorders.

### DETAILED DESCRIPTION OF THE INVENTION

In accordance with the present invention, there are provided pharmaceutical compositions and combinations comprising a non-immunosuppressive cyclosporin and methods of treating Flaviviridae viral infections and induced disorders using the same.

Flaviviruses included within the scope of this invention are discussed generally in Fields Virology, Editors: Fields, N., Knipe, D. M. and Howley, P. M.; Lippincott-Raven Publishers, Philadelphia, Pa.; Chapter 31 (1996). Specific flaviviruses include, without limitation: Absettarov; Alfuy; Apoi; Aroa; Bagaza; Banzi; Bououi; Bussuquara; Cacipacore; Carey Island; Dakar bat; Dengue viruses 1, 2, 3 and 4; Edge Hill; Entebbe bat; Gadgets Gully; Hanzalova; Hypr; Ilheus; Israel turkey meningoencephalitis; Japenese encephalitis; Jugra; Jutiapa; Kadam; Karshi; Kedougou; Kokoera; Koutango; Kumlinge; Kunjin; Kyasanur Forest disease; Langat; Louping ill; Meaban; Modoc; Montano myotis leukoencephalitis; Murray valley encephalitis; Naranjal; Negishi; Ntaya; Omsk Hemorrhagic fever; Phnom-penh bat; Powassan; Rio Bravo; Rocio; Royal Farm; Russian spring-summer encephalitis; Saboya; St. Louis encephalitis; Sal Vieja; San Perlita; Saumarex Reef; Sepik; Sokuluk; Spondweni; Stratford; Temusu; Tyuleniy; Uganda S, Usutu, Wesselsbron; West Nile; Yaounde; Yellow fever; and Zika.

Pestiviruses included within the scope of this invention are also discussed generally in Fields Virology (Id.). Specific pestiviruses include, without limitation: bovine viral diarrhea virus (“BVDV”); classical swine fever virus (“CSFV”) also known as hog cholera virus); and border disease virus (“BDV”).

Other members of the Flaviviridae family within the scope of the present invention include but are not limited to GB virus A, GB virus A-like agents, GB virus-B and GB virus-C (also called hepatitis G virus, HGV). In addition, the hepacivirus group (hepatitis C virus; HCV), including the approximately 6 HCV genotypes and more than 50 subtypes are also within the scope of the present invention.

A cyclosporin is considered as binding to cyclophilin if it binds to human recombinant cyclophilin at least one fifth as well as does cyclosporin A in the competitive ELISA test described by Quesniaux in Eur. J. Immunol. 1987 17 1359-1365. In this test, the cyclosporin to be tested is added during the incubation of cyclophilin with coated BSA-cyclosporin A and the concentration required to give a 50% inhibition of the control reaction without competitor is calculated (IC₅₀). The results are expressed as the Binding Ratio (BR), which is the log to the base 10 of the ratio of the IC₅₀ of the test compound and the IC₅₀ in a simultaneous test of cyclosporin A itself. Thus a BR of 1.0 indicates that the test compound binds human cyclophilin one factor of ten less well than does cyclosporin A, and a negative value indicates binding stronger than that of cyclosporin A. The cyclosporins active against HCV have a BR lower than 0.7, preferably equal to or lower than zero.

Examples of non immunosuppressive cyclophilin-binding cyclosporins include e.g. compounds of Formula I

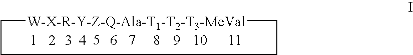

wherein
W is MeBmt, dihydro-MeBmt, 8′-hydroxy-MeBmt or O-acetyl-MeBmt¹;
   X is αAbu, Val, Thr, Nva or 0-methyl threonine (Me0Thr);
   R is Pro, Sar, (D)-MeSer, (D)-MeAla, or (D)-MeSer(Oacetyl);
   Y is MeLeu, thioMeLeu, γ-hydroxy-MeLeu, MeIle, MeVal, MeThr, MeAla, MeaIle or MeaThr; N-ethylVal, N-ethylIle, N-ethylThr, N-ethylPhe, N-ethylTyr or N-ethylThr(Oacetyl)
   Z is Val, Leu, MeVal or MeLeu,
   Q is MeLeu, γ-hydroxy-MeLeu, MeAla or Pro,
   T₁ is (D)Ala or Lys,
   T₂ is MeLeu or γ-hydroxy-MeLeu, and
   T₃ is MeLeu or MeAla.

Preferred compounds of formula I are e.g. compounds of formula Ia

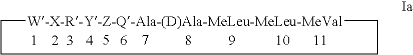

in which
W′ is MeBmt, dihydro-MeBmt or 8′-hydroxy-MeBmt;
X is αAbu, Val, Thr, Nva or 0-methyl threonine (Me0Thr);
R′ is Sar, (D)-MeSer, (D)-MeAla, or (D)-MeSer(Oacetyl);
Y′ is MeLeu, γ-hydroxy-MeLeu, MeIle, MeVal, MeThr, MeAla, MeaIle or MeaThr; N-ethylVal, N-ethylIle, N-ethylThr, N-ethylPhe, N-ethylTyr or N-ethylThr(Oacetyl)
Z is Val, Leu, MeVal or MeLeu; and
Q′ is MeLeu, γ-hydroxy-MeLeu or MeAla.

The groups W′, X, Y′, Z, Q′ and R′ have, independently, the following preferred significances:
W′ is preferably W″ where W″ is MeBmt or dihydro-MeBmt;
X is preferably X′ where X′ is αAbu or Nva, more preferably X″ where X″ is αAbu;
R′ is preferably R″ where R″ is Sar;
Y′ is preferably Y″ where Y″ is γ-hydroxy-MeLeu, MeVal, MeThr, MeIle, N-ethylIle or N-ethylVal;
Z is preferably Z′ where Z′ is Val or MeVal; and
Q′ is preferably Q″ where Q″ is MeLeu;

A preferred group of Compounds of formula Ia are those in which W′ is W″, X is X′, Y′ is Y″, Z is Z′, Q′ is Q″ and R′ is R″.

Examples of preferred compounds of Formula Ia are e.g.:
a) [dihydro-MeBmt]¹-[γ-hydroxy-MeLeu]⁴-Ciclosporin; BR*=0.1; IR<1%
b) [MeVa]⁴-Ciclosporin; BR=0.1; IR<1%
c) [Melle]⁴-Ciclosporin; BR=−0.2; IR<1%
d) [MeThr]⁴-Ciclosporin;
e) [γ-hydroxy-MeLeu]⁴-Ciclosporin; BR=0.4; IR<1%
f) [Ethyl-Ile]⁴-Ciclosporin; BR=0.1; IR<2%
g) [Ethyl-Val]⁴-Ciclosporin; BR=0; IR<2%
h) [Nva]²-[γ-hydroxy-MeLeu]⁴-Ciclosporin;
i) [γ-hydroxy-MeLeu]⁴-[γ-hydroxy-MeLeu]⁶-Ciclosporin;
j) [MeVal]⁵-Ciclosporin; BR=0.4; IR=5.3%
k) [Me0Thr]²-[(D)MeAla]³-[MeVal]⁵-Ciclosporin;
l) [8′-hydroxy-MeBmt]¹-Ciclosporin; BR=0.35; IR=1.8%

k) [MeAla]⁶-Ciclosporin; BR=−0.4; IR=3.2

l) [γ-hydroxy-MeLeu]⁹-Ciclosporin; BR=0.15; IR=2.9

IR=immunosuppressive Ratio, expressed as a percentage of the activity relative to Cyclosporin A.

Further examples of non-immunosuppressive cyclosporins are the compounds disclosed in WO 98/28330, WO 98/28329 and WO 98/28328, the contents thereof being incorporated herein by reference, e.g. compounds of formula II

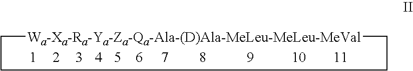

wherein

### Wₐ is

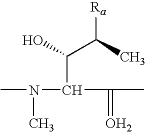

wherein Rₐ is a residue of formula Ic or Id

—CH₂—CH═CH—CH₂—R₄ Ic or —CH₂—SH—R′₄ Id

in which R₄ is C₁₋₄alkylthio, aminoC₁₋₄alkylthio, C₁₋₄alkylaminoC₁₋₄alkylthio, diC₁₋₄alkylamino-C₁₋₄alkylthio, pyrimidinylthio, thiazolylthio, N—C₁₋₄alkylimidazolylthio, hydroxyC₁₋₄alkylphenylthio, hydroxyC₁₋₄alkylphenoxy, nitrophenylamino or 2-oxopyrimidin-1-yl, and R′₄ is C₁₋₄ alkyl,

### Xₐ is Abu;

Rₐ is —NMe-CH(R_{b})—CO— wherein R_{b} is H or —S-Alk-R₀ in which Alk-R₀ is methyl; or Alk is straight or branched C₂₋₆alkylene or C cycloalkylene and R₀ is H; OH; COOH; C₂₋₅alkoxy-carbonyl; NR₁R₂ in which each of R₁ and R₂, independently, is selected from H, C₁₋₄alkyl, C₂₋₄alkenyl, C₃₋₆cycloalkyl and phenyl each optionally substituted by halogen, C₁₋₄alkoxy, C₂₋₅alkoxycarbonyl, amino, C₁₋₄alkylamino and/or diC₁₋₄alkyl-amino, and benzyl and a heterocyclic radical, said benzyl and heterocyclic radicals being saturated or unsaturated and containing 5 or 6 ring members and 1 to 3 heteroatoms, or R₁ and R₂ form, together with the nitrogen atom to which they are attached, a 4- to 6 membered heterocycle which may contain another heteroatom chosen from nitrogen, oxygen and sulphur, and which is optionally substituted by C₁₋₄alkyl, phenyl or benzyl; or each of R₁ and R₂, independently, is a radical of formula Ib

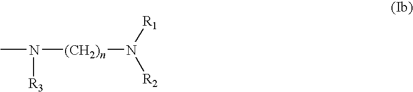

in which R₁ and R₂ are as defined above, R₃ is H or C₁₋₄alkyl and n is an integer ranging from 2 to 4;
Yₐ is MeLeu or γ-hydroxy-MeLeu;

### Zₐ is Val; and

### Qₐ is MeLeu,

with the proviso that R_{b} is not H when Y, is MeLeu,
or a pharmaceutically acceptable salt thereof.

In the formula II, when R₁ and/or R₂ is a heterocyclic residue, it may be pyridyl, tetrahydro-pyridyl, piperidyl, imidazolyl, oxazolyl or thiazolyl. When R₁ and R₂ form a heterocyclic residue with the nitrogen atom to which they are attached, by way of example, the heterocyclic residue may be chosen from azetidinyl, piperidyl, piperazinyl, N-methyl-piperazinyl, N-phenylpiperazinyl, N-benzylpiperazinyl, pyridyl, imidazolyl, morpholino, thiomorpholino, tetrahydropyridyl, methyltetrahydropyridyl (for example 4-methyl-tetrahydropyridyl) or phenyltetrahydropyridyl (for example 4-phenyltetrahydropyridyl).

The Compounds of formula I, Ia or II may be obtained in a variety of ways, which may be classified as:

1) Fermentation

2) Biotransformation

3) Derivatisation

4) Partial Synthesis

5) Total Synthesis

as disclosed e.g. in EP 0 484 281 A1, WO 00101715, WO 98/28330, WO 98/28329 or WO 98/28328 the contents thereof being incorporated herein by reference.

In a series of further specific or alternative embodiments, the present invention also provides:
1.1 A method for preventing or treating Flaviviridae infections or Flaviviridae induced disorders in a subject in need thereof, comprising administering to said subject a therapeutically effective amount of a non-immunosuppressive cyclophilin-binding cyclosporin, e.g. a compound of formula I, Ia or II.
   According to the invention, the non-immunosuppressive cyclophilin-binding cyclosporine may be administered in an amount effective to alleviate or eliminate one or more of the signs or symptoms of Flaviviridae infection or induced disorder, for example, effective to lower the Flaviviridae virus measured in a serum sample of a subject.
1.2 A method for inhibiting Flaviviridae replication in a medium, comprising applying to this medium an effective amount of a non-immunosuppressive cyclophilin-binding cyclosporin, e.g. a compound of formula I, Ia or II.
1.3 A method for inhibiting Flaviviridae replication in a subject in need thereof, comprising administering to this subject a therapeutically effective amount of a non-immunosuppressive cyclophilin-binding cyclosporin, e.g. a compound of formula I, Ia or I.
1.4 A method for preventing the recurrence of HCV infection in a transplant recipient in need thereof, comprising administering to said recipient a therapeutically effective amount of a non-immunosuppressive cyclophilin-binding cyclosporin, e.g. a compound of formula I, Ia or II.
2. Use of a non-immunosuppressive cyclophilin-binding cyclosporin, e.g. a compound of formula I, Ia or II, in the preparation of a pharmaceutical composition for use in any method as defined above.
3. A pharmaceutical composition for use in any method as defined above, comprising a non-immunosuppressive cyclophilin-binding cyclosporin, e.g. a compound of formula I, Ia or II, together with one or more pharmaceutically acceptable diluents or carriers therefor.

Utility of the non-immunosuppressive cyclophilin-binding cyclosporins (hereinafter “cyclosporins of the invention” or “subject non-immunosuppressive cyclosporin”) in treating diseases and conditions as hereinabove specified may be demonstrated in standard animal or clinical tests, e.g. in accordance with the methods described hereinafter.

A. In Vitro

Cell culture: Huh-7 and MH-14 cells, HCV replicon cells, are cultured in Dulbecco's modified Eagle's medium (DMEM) with 10% fetal bovine serum (FBS). PH5CH8 cells are cultured in a 1:1 mixture of DMEM and F12 medium supplemented with 100 ng/ml of epidermal growth factor, 10 μ/ml of insulin, 0.36 μg/ml of hydrocortisone, 5 μg/ml of transferrin, 5 μg/ml of linoleic acid, 20 ng/ml of selenium, 4 μg/ml of glucagon, 10 ng/ml of prolactin, 10 μg/ml of gentamicin, 200 μg/ml of kanamycin, and 2% FBS.

Immunoblot analysis: Immunoblot analysis is performed as described by K. Watashi et al., Virology 2001, 286, 391-402. The primary antibodies used in this experiment are anti-NS5A, anti-NS5B, and anti-β-actin (Sigma) antibodies.

Indirect immunofluorescence analysis: Indirect immunofluorescence analysis is performed as described by K. Watashi, supra. The primary antibodies used in this experiment are anti-NS5A and anti-PDI (StressGen) antibodies.

### Reverse Transcription (RT)-Polymerase Chain Reaction (PCR) Analysis

Total RNA of cultured cells is isolated with Sepasol-RNA I Super (nacalai tesque) as recommended by the manufacturer. RT-PCR analysis is performed using a one step RNA PCR kit (Takara) according to the manufacturer's directions. The primers used for detection of mRNAs for 2′,5′-oligoadenylate synthetase and double strand RNA-dependent protein kinase are 5′-CCGTGAAGTTTGAGGTCCAG-3′,5′-GACTAATTCCAAGACCGTCCG-3′ and 5′-TGGCCGCTAAACTTGCATATC-3′,5′-GCGAGTGTGCTGGTCACTAAAG-3′, respectively.

Northern blot analysis: Northern blot analysis is performed as described by H. Kishine et al., Biochem. Biophys. Res. Commun., 2002, 47, 119-125. The probe complementary to the NS5B sequence used in this experiment is described by H. Kishine, supra.

Real time RT-PCR analysis: The 5′-UTR of HCV genome RNA is quantified using the ABI PRISM 7700 sequence detector (AppliedBiosystems) as described by T. Takeuchi et al., Gastroenterology, 1999, 116, 636-642. The forward and reverse primers used in this experiment are 5′-CGGGAGAGCCATAGTGG-3′ and 5′-AGTACCACAAGGCCTTTCG-3′, respectively. The fluorogenic probe is 5′-CTGCGGAACCGGTGAGTACAC-3′. As an internal control, ribosomal RNA is also quantified using TaqMan Ribosomal RNA Control Reagents (Applied Biosystems).

In vitro HCV infection experiment: The in vitro HCV infection experiment is performed essentially as described by N. Kato et al., Jpn. J. Cancer Res. 1996, 87, 787-792 and M. Ikada et al., Virus Res., 1998, 56, 157-167. PH5CH8 cells (1×10⁵) are infected with the plasma 1B-2 (equivalent to 10⁴ to 10⁵ HCV RNA copies), which is prepared from an HCV-positive blood donor. At 24 h post-inoculation, the cells are washed three times with phosphate-buffered saline (PBS) and maintained with fresh medium.

Transfection and reporter assay: Transfection into MH-14 and H9 cells is performed using FuGENE 6 (Roche) and Lipofectamine 2000 transfection reagent (Invitrogen), respectively, according to the manufacturer's protocol. The reporter assay is performed as described by K. Watashi, supra. The reporter plasmids used in this study are pNFAT-Luc, pAPi-Luc, pNFKB-Luc (PathDetect Reporter System; Stratagene), and pRL-TK (Dual-luciferase reporter assay system; Promega).

The effect of various cyclosporins of the invention on the replication of the HCV genome using MH-14 cells, in which the HCV subgenomic replicon as shown in FIG. 1A is autonomously replicated. Treatment with a cyclosporin of the invention, e.g. [Melle]⁴-ciclosporin, e.g. at 1 μg/ml, as well as 100 U/ml IFNα which is used as a positive control for 7 days decreases the amount of HCV NS5A and NS5B proteins to levels undetectable by immunoblot analysis. Indirect immunofluorescence analysis showed that NS5A protein production is reduced in all the cells treated with 1 μg/ml cyclosporin of the invention, while the level of protein disulfide isomerase (PDI), which is an endoplasmic reticulum marker, as an internal control is not altered under this condition. The cyclosporins of the invention decrease in this assay HCV protein expression in HCV replicon cells.

Replicon RNA is analyzed in MH-14 cells treated with or without a cyclosporin of the invention or IFNα for 7 days by northern blot analysis. Treatment with e.g. 1 μg/ml cyclosporin of the invention, e.g. [Melle]⁴-ciclosporin, decreases the amount of replicon RNA to an undetectable level. Treatment with 100 U/ml IFNα produces a similar effect. In addition the titer is gradually decreased and the level of HCV RNA is reduced to about 1/400 of the original on the 7^{th} day. In the case of a co-treatment with IFNα, a further reduction at any time point examined (3th, 5^{th} and 7^{th} day) compared with the single treatment with either the cyclosporin or IFNα: the replicon RNA level in MH-14 cells treated with both the cyclosporin and IFNα for 7 days is significantly decreased over that in the cells treated with IFNα alone.

Furthermore, PH5CH8 cells (non-neoplastic hepatocyte cell line) are treated with HCV-positive plasma and subsequently the HCV RNA genome titer at various time-points post-inoculation is quantified by real time RT-PCR analysis. While the HCV RNA genome titer on the 5^{th} day post-inoculation in the cells is increased about 10-fold compared with that on the 1^{st} day, a significant increase of the HCV RNA genome titer at these time points was not observed in the cells treated continuously with a cyclosporin of the invention, e.g. [Melle]⁴-ciclosporin, or IFNα. The cyclosporins of the invention inhibit the replication of HCV infected cultured hepatocytes.

Results are shown in FIGS. 2E, 2F and 2G: immunoblot analysis (2E), indirect immunofluorescence analysis (2F) and real time RT-PCR analysis (2G) is performed using MH-14 cells treated with [Melle]⁴-Ciclosporin (▪) or an non cyclophilin binding cyclosporin (), e.g. 6-[[R-(E)]6,7-Didehydro-N, 4-dimethyl-3-oxo-L-2-aminooctanoic acid]-7-L-valine-cyclosporin A. Control in 2E and 2F (1^{st} row), no treatment; CysA in 2E, 1 μg/ml; [Melle]⁴-Ciclosporin in 2E (▪) and 2F (▪), 1 g/ml; the non cyclophilin binding cyclosporin in 2E (▪) and 2F (▪), 1 μg/ml.

### Further Cell Culture Systems for Determining Antiviral Activities

The methods described above and some of the methods described below utilize HCV. The described methods however, may be adapted to other members of the Flaviviridae family simply by changing the cell system and viral pathogen.

A useful cell-based assay to detect HCV and its inhibition assesses the levels of replicon RNA from Huh7 cells harbouring the HCV replicon. These cells can be cultivated in standard media, for example DMEM medium (high glucose, no pyruvate), supplemented with 10% fetal bovin serum, 1× non-essential amino acids, Pen-Strep-Glu (100 units/liter, 100 microgram/liter, and 2.92 mg/liter, respectively), and G418 (500 to 1000 microgram/millilitre). Antiviral screening assays can be done in the same medium without G418. To keep the cells in the logarithmic growth phase, cells are seeded in 96-well plates at low density, as for example, 1000 cells per well. The test compound i.e., a subject non-immunosuppressive cyclosporin, is then added immediately after seeding the cells and they are incubated for 3 to 7 days at 37° C. in an incubator. The medium is then removed, and the cells prepared for total RNA extraction (replicon RNA+host RNA). Replicon RNA can then be amplified in a real-time RT-PCR (Q-RT-PCR) protocol, and quantified.

The observed differences in quantification of replicon RNA are one way to express the antiviral potency of the test compound, i.e., a subject non-immunosuppressive cyclosporin. In a typical experiment, a comparable amount of replicon is produced in the negative control and with non-active compounds. This can be concluded if the measured threshold-cycle for the flavivirus or pestivirus RT-PCR in both settings is approximately the same. In such experiments, a way to express the antiviral effectiveness of a compound is to subtract the average threshold RT-PCR cycle of the negative control (Ct_{negative}) from the threshold RT-PCR cycle of the test compound (Ct_{testcompound}). This value is called [symbol] Ct ( [symbol] Ct=Ct_{testcompound}−Ct_{negative}). A [symbol] Ct value of 3.3 represents a 1-log reduction in replicon production. As a positive control, recombinant interferon alpha-2a (for example, Roferon-A, Hoffmann-Roche, N.J., USA) can be used alongside the test compound, i.e., a subject non-immunosuppressive cyclosporin. Furthermore, the compounds can be tested in dilution series (typically at 100, 33, 10 3 and 1:M). The [symbol] Ct values for each concentration allow the calculation of the 50% effective concentration (EC₅₀).

As discussed earlier, the assays described above can be adapted to the other members of the Flaviviridae by changing the cell system and the viral pathogen. Methodologies to determine the efficacy of a subject non-immunosuppressive cyclosporin include modifications of the standard techniques as described by Holbrook M R et al. Virus Res. 2000, 69, 31; Markland W. et al. Antimicrob. Agents. Chemother. 2000, 44, 859; Diamond M S et al., J. Virol. 2000, 74, 7814; Jordan I et al. J. Infect Dis. 2000, 182, 1214; Sreenivasan V et al. J. Virol. Methods 1993, 45 (1), 1; or Baginski S G et al. Proc. Natl. Acad. Sci USA 2000, 97 (14) 7981 or the real-time RT-PCR technology. As an example, an HCV replicon system in HuH7 cells (Lohmann V et al. Science, 1999, 285 (5424, 110) or modifications thereof (Blight et al. 2000) can be used.

### Cell Protection Assay

An assay may be performed essentially as described by Baginski, S. G., et al. “Mechanism of action of a pestivirus compound” PNAS USA 2000, 97(14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of a subject non-immunosuppressive cyclosporin may be added to both infected and uninfected cells in a final concentration of 0.5% DMSO in growth medium. Each dilution may be tested in duplicate, triplicate or quadruplicate. Cell densities and virus inocula can be adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells may be read in a microplate reader at 550 nm. The 50% effective concentration (EC₅₀) values are defined as the concentration of a subject non-immunosuppressive cyclosporin that achieved 50% reduction of cytopathic effect of the virus.

### Plaque Reduction Assay

Effective concentrations for a subject non-immunosuppressive cyclosporin may be determined in duplicate 24-well plates by plaque reduction assays. Cell monolayers are infected with 100 PFU/well of virus. Then, serial dilutions of a subject non-immunosuppressive cyclosporin in MEM supplemented with 2% inactivated serum and 0.75% of methyl cellulose are added to the monolayers. Cultures are further incubated at 37 C for 3 days, then fixed with 50% ethanol and 0.8% Crystal Violet, washed and air-dried. Plaques are counted to determine the concentration to obtain 90% virus suppression.

### Yield Reduction Assay

For each subject non-immunosuppressive cyclosporin, the concentration to obtain a 6-log reduction in viral load may be determined in duplicate 24-well plates by yield reduction assays. The assay may be performed as described in Baginski, S. G., et al. “Mechanism of action of a pestivirus compound” PNAS USA 2000, 97(14), 7981-7986, with minor modifications. MDBK cells are seeded onto 24-well plates (2×10⁵ cells per well) 24 hours before infection with BVDV (NADL strain) at a multiplicity of infection (MOI) of 0.1 PFU per cell. Serial dilutions of non-immunosuppressive cyclosporins are added to cells in a final concentration of 0.5% DMSO in growth medium. Each dilution may be tested in duplicate, triplicate, or quadruplicate. After three days, cell cultures (cell monolayers and supernatants) are lysed by multiple freeze-thaw cycles, and virus yield quantified by plaque assay. Briefly, MDBK cells are seeded onto 6-well plates (5×105 cells per well) 24 hours prior to use. Cells are inoculated with 0.2 mL of test lysates for 1 hour, washed and overlaid with 0.5% agarose in growth medium. After 3 days, cell monolayers are fixed with 3.5% formaldehyde and stained with 1% crystal violet (w/v in 50% ethanol) to visualize plaques. The plaques are then counted to determine the concentration to obtain a 6-log reduction in viral load.

### Non-Cell Based Assays Adapted for Detecting Flaviviridae Viruses

Nucleic acid amplification technology is now the method of choice for identification of a large and still growing number of microorganisms such as Mycobacterium tuberculosis, human immunodeficiency virus (HIV), and hepatitis C virus (HCV) in biological samples. Nucleic acid amplification techniques include the polymerase chain reaction (PCR), ligase chain reaction (LCR), nucleic acid sequence-based amplification (NASBA), strand-displacement amplification (SDA), and transcription-mediated amplification (TMA). The nucleotide sequence for at least a portion of a Flaviviridae virus genome is required for nucleic acid amplification techniques. Such sequences are readily available in the published literature and genetic databases.

### Amplified-Product Detection Schemes

Amplified-product detection schemes are of two basic types: heterogeneous and homogeneous. Heterogeneous detection is characterized by a distinct step, such as washing, designed to remove unhybridized probes from hybridised probes, whereas in homogeneous detection there is no physical separation step to remove free probe from bound probe. Multiple heterogeneous and homogeneous detection methods exist.

### Heterogeneous Detection

Southern blotting, for example, is a heterogeneous detection technique. In Southern blotting, electrophoresis is used to separate amplification products by size and charge. The size-fractionated products are transferred to a membrane or filter by diffusion, vacuuming, or electroblotting. Labelled detection probes are then hybridised to the membrane-bound targets in solution, the filters are washed to remove any unhybridized probe, and the hybridised probe on the membrane is detected by any of a variety of methods.

Other types of heterogeneous detection are based on specific capture of the amplification products by means of enzyme-linked immunosorbent assays (ELISAs). One method used with PCR involves labelling one primer with a hapten or a ligand, such as biotin, and, after amplification, capturing it with an antibody- or streptavidin-coated microplate. The other primer is labelled with a reporter such as fluorescein, and detection is achieved by adding an antifluorescein antibody, horseradish peroxidase (HRP) conjugate. This type of method is not as specific as using detection probes that hybridise to defined amplification products of interest.

### Homogeneous Detection

Because hybridised and nonhybridized detection probes are not physically separated in homogeneous detection systems, these methods require fewer steps than heterogeneous methods and thus are less prone to contamination. Among the commercially available kits that use homogeneous detection of fluorescent and chemiluminescent labels are the TaqMan system (Applied Biosystems; Foster City, Calif.), BDProbeTecET system (Becton Dickinson; Franklin Lakes, N.J.), QPCR System 5000 (Perkin-Elmer Corp.; Norwalk, Conn.) and Hybridization Protection Assay (Gen Probe Inc.; San Diego).

The TaqMan system detects amplicon in real time. The detection probe, which hybridizes to a region inside the amplicon, contains a donor fluorophore such as fluorescein at its 5′ end and a quencher moiety, for example, rhodamine, at its 3′ end. When both quencher and fluorophore are on the same oligonucleotide, donor fluorescence is inhibited. During amplification the probe is bound to the target. Taq polymerase displaced and cleaves the detection probe as it synthesizes the replacement strand. Cleavage of the detection probe results in separation of the fluorophore from the quencher, leading to an increase in the donor fluorescence signal. During each cycle of amplification the process is repeated. The amount of fluorescent signal increases as the amount of amplicon increases.

Molecular beacons use quenchers and fluorophores also. Beacons are probes that are complementary to the target amplicon, but contain short stretches (approximately 5 nucleotides) of complementary oligonucleotides at each end. The 5′ and 3′ ends of the beacons are labelled with a fluorophore and a quencher, respectively. A hairpin structure is formed when the beacon is not hybridised to a target, bringing into contact the fluorophore and the quencher and resulting in fluorescent quenching. The loop region contains the region complementary to the amplicon. Upon hybridization to a target, the hairpin structure opens and the quencher and fluorophore separate, allowing development of a fluorescent signal. A fluorometer measures the signal in real time.

The BDProbeTecET system uses a real-time detection method that combines aspects of Taq Man and molecular beacons. The probe has a hairpin loop structure and contains fluorescein and rhodamine labels. In this system, however, the region complementary to the target molecule is not within the loop but rather in the region 3′ to the rhodamine label. Instead of containing the sequence complementary to the target, the single-stranded loop contains a restriction site for the restriction enzyme BsoBI. The single-stranded sequence is not a substrate for the enzyme. The fluorescein and rhodamine labels are near each other before amplification, which quenches the fluorescein fluorescence. Strand-displacement amplification converts the probe into a double-stranded molecule. The BsoBI restriction enzyme can then cleave the molecule, resulting in separation of the labels and an increase in the fluorescent signal.

The QPCR System 5000 employs electrochemiluminescence with ruthenium labels. A biotinylated primer is used. After amplification, the biotin products are captured on streptavidin-coated paramagnetic beads. The beads are transferred into an electrochemical flow cell by aspiration and magnetically held to the surface of the electrode. Upon electrical stimulation, the ruthenium-labeled probe emits light.

Detection-probe design is critical in all methodologies that use probes to detect amplification products. Good detection probes hybridise only to specified amplification product and do not hybridise to non-specific products. Other key issues in optimising detection methodologies involve the labelling of probes and the maximization of sample throughput.

### Labelling Methods and Reporter Molecules

Detection probes can be labeled several different ways. Enzymatic incorporation of ³²P or ³⁵S into the probes is the most common method for isotopical labeling. Following hybridisation and washing, the signal is detected on autoradiographic film.

To perform nonradioactive detection, probes can be enzymatically labelled with a variety of molecules. Biotin can be incorporated enzymatically and then detected with streptavidin-conjugated alkaline phosphatase, using AP substrates like 5-bromo-4-chloro-3-indolyl phosphate (BCIP) and nitroblue tetrazolium (NBT). Chemiluminescent substrates such as Lumi-Phos 530 or Lumi-Phos Plus (Lumigen, Southfield, Mich.) can also be used with AP. In addition, digoxigenin-11-dUTP can be incorporated enzymatically into DNA or RNA, and antidigoxigenin AP conjugates can be used with calorimetric or chemiluminescent detection.

There are numerous other types of reporter molecules, including chemiluminescent moieties such as acridinium esters. Many fluorescent moieties are available as well. Electrochemiluminescent compounds such as tris (2,2′-bipyridine) ruthenium (II) can be used also. Further discussions of these and similar techniques can be found in: Schiff E R de Medina M, Kahn R S. Semin Liver Dis. 1999; 19 (Suppl 1:3-15).

Any of these heterogeneous or homogeneous assays may be utilized to assess the effectiveness of the cyclosporins of the invention against a virus of the Flaviviridae family.

B. Clinical Trial

A total of 15 patients with chronic Hepatitis C infection or other Flaviviridae virus infection, are enrolled in a study of 2 weeks. Each patient receives a cyclosporine of the invention, e.g. [Melle]⁴-ciclosporin, at a dose of 7 to 15 mg/kg p.o. The serum levels of Hepatitis C antigens (or other Flaviviridae virus antigens) are determined at day 0 and day 14 in each patient.

A person suffering from hepatitis C infection may exhibit one or more of the following signs or symptoms: (a) elevated ALT, (b) positive test for anti-HCV antibodies, (c) presence of HCV as demonstrated by a positive test for HCV-RNA, (d) clinical stigmata of chronic liver disease, (e) hepatocellular damage. Such criteria may not only be used to diagnose Hepatitis C, but can be used to evaluate a patient's response to drug treatment.

Elevated serum alanine aminotransferase (ALT) and aspartate aminotransferase (AST) are known to occur in uncontrolled Hepatitis C, and a complete response to treatment is generally defined as the normalization of these serum enzymes, particularly ALT (Davis et al., 1989, New Eng. J. Med. 321:1501-1506). ALT is an enzyme released when liver cells are destroyed and is symptomatic of HCV infection.

In order to follow the course of HCV replication or other Flaviviridae virus replication in subjects in response to drug treatment, the virus RNA may be measured in serum samples by, for example, a nested polymerase chain reaction assay that uses two sets of primers derived from the N53 and N54 non-structural gene regions of the HCV genome (Farci et al., 1991, New Eng. J. Med. 325:98-104. Ulrich et al., 1990, J. Clin. Invest., 86:1609-1614) or similar region of another Flaviviridae virus.

Histological examination of liver biopsy samples may be used as a second criteria for evaluation of HCV replication. See, e.g., Knodell et al., 1981, Hepatology 1:431-435, whose Histological Activity Index (portal inflammation, piecemeal or bridging necrosis, lobular injury and fibrosis) provides a scoring method for disease activity.

Daily dosages required in practicing the method of the present invention will vary depending upon, for example, the non-immunosuppressive cyclophilin-binding cyclosporin employed, the host, the mode of administration, the severity of the condition to be treated. A preferred daily dosage range is about from 1 to 50 mg/kg per day as a single dose or in divided doses. Suitable daily dosages for patients are on the order of from e.g. 1 to 20 mg/kg p.o or i.v. Suitable unit dosage forms for oral administration comprise from ca. 0.25 to 10 mg/kg active ingredient, e.g. [Melle]⁴-ciclosporin, together with one or more pharmaceutically acceptable diluents or carriers therefor.

The cyclosporins of the invention may be administered by any conventional route, in particular enterally, e.g. orally, for example in the form of solutions for drinking, tablets or capsules or parenterally, for example in the form of injectable solutions or suspensions. Preferred pharmaceutical compositions may be e.g. those based on microemulsions as described in UK 2,222,770 A.

The cyclosporins of the invention may be administered as the sole ingredient or together with other drugs, e.g. a drug which has anti-Flaviviridae activities, e.g. an interferon, e.g. interferon-α-2a or interferon-α-2b, e.g. Intron^{R A}, Roferon^{R}, Avonex^{R}, Rebif^{R} or Betaferon^{R}, or an interferon conjugated to a water soluble polymer or to human albumin, e.g. albuferon, an anti-viral agent, e.g. ribavirin, lamivudine, NV08 or NM283, an inhibitor of the HCV or other Flaviviridae virus encoded factors like the NS3/4A protease, helicase or RNA polymerase or a prodrug of such an inhibitor, an anti-fibrotic agent, e.g. a N-phenyl-2-pyrimidine-amine derivative, e.g. imatinib, an immune modulating agent, e.g. mycophenolic acid, a salt or a prodrug thereof, e.g. sodium mycophenolate or mycophenolate mofetil, or a S1P receptor agonist, e.g. FTY720 or an analogue thereof optionally phosphorylated, e.g. as disclosed in EP627406A1, EP778263A1, EP1002792A1, WO02/18395, WO02/76995, WO 02/06268, JP2002316985, WO03/29184, WO03/29205, WO03/62252 and WO03/62248.

Conjugates of interferon to a water-soluble polymer are meant to include especially conjugates to polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236 987, European Patent Application No. 0 510 356 and international Application Publication No. WO 95/13090. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-α-2a, pegylated interferon-α-2b; pegylated consensus interferon or pegylated purified interferon-α product. Pegylated interferon-α-2a is described e.g. in European Patent 593,868 and commercially available e.g. under the tradename PEGASYS® (Hoffmann-La Roche). Pegylated interferon-α-2b is described, e.g. in European Patent 975,369 and commercially available e.g. under the tradename PEG-INTRON A® (Schering Plough). Pegylated consensus interferon is described in WO 96/11953. The preferred pegylated α-interferons are pegylated interferon-α-2a and pegylated interferon-α-2b. Also preferred is pegylated consensus interferon.

Ribavirin (1-β-D-ribofuranosyl-1-1,2,4-triazole-3-caroxamide) is a synthetic, non-interferon-inducing, broad spectrum antiviral nucleoside analog sold under the trade name, Virazole (The Merk Index, 11^{th} edition, Editor: Budavar, S, Merck & Co., Inc., Rahway, N.J., p 1304, 1989). U.S. Pat. No. 3,798,209 and RE29,835 disclose and claim ribavirin. Ribavirin is structurally similar to guanosine, and has in vitro activity against several DNA and RNA viruses including Flaviviridae (Gary L. Davis, Gastroenterology 118:S104-S114, 2000).

Ribavirin reduces serum amino transferase levels to normal in 40% of patients, but it does not lower serum levels of HCV-RNA (Gary L. Davis, Gastroenterology 118:S104-S114, 2000). Thus, ribavirin alone is not effective in reducing viral RNA levels. Additionally, ribavirin has significant toxicity and is known to induce anemia. Ribavirin is not approved for monotherapy against HCV; it is approved in combination with interferon alpha-2a or interferon alpha-2b for the treatment of HCV.

Daily dosages with respect to the co-agent used will vary depending upon, for example, the compound employed, the host, the mode of administration and the severity of the condition to be treated. For example, lamivudine may be administered at a daily dosage of 100 mg. The pegylated interferon may be administered parenterally one to three times per week, preferably once a week, at a total weekly dose ranging from 2 to 10 million IU, more preferable 5 to 10 million IU, most preferable 8 to 10 million IU.

In accordance with the foregoing the present invention provides in a yet further aspect
4. A pharmaceutical combination comprising a) a first agent which is a non-immunosuppressive cyclophilin-binding cyclosporin, e.g. a compound of formula I, Ia or II, and b) a co-agent, e.g. a second drug agent as defined above, e.g. for use in any method as defined above.
5. A method as defined above comprising co-administration, e.g. concomitantly or in sequence, of a therapeutically effective amount of a non-immunosuppressive cyclophilin-binding cyclosporin, e.g. a compound of formula I, Ia or II, and a co-agent, e.g. a second drug agent as defined above.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The administration of a pharmaceutical combination of the invention results in a beneficial effect, e.g. a synergistic therapeutic effect, compared to a monotherapy applying only one of its pharmaceutically active ingredients. A preferred synergistic combination is a combination of a non-immunosuppressive cyclophilin-binding cyclosporin with an interferon, optionally conjugated to a polymer.

A further preferred combination is a combination of a non-immunosuppressive cyclophilin-binding cyclosporin with mycophenolic acid, a salt or a prodrug thereof, or with a SIP receptor agonist, e.g. FTY720.

### Combination or Alternation Therapy

The active compounds of the present invention can be administered in combination or alternation with another anti-Flaviviridae agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In preferred embodiments, an anti-Flaviviridae compound that exhibits an EC₅₀ of 10-15:M, or preferably less than 1-5:M, is desirable.

It has been recognized that drug-resistant variants of Flaviviridae viruses can emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameters of the drug can be altered by such combination or alternation therapy. In general, combination therapy is typically preferred over alternation therapy because it induces multiple simultaneous stresses on the virus.

As described above, a number of viral treatments e.g., interferons and Ribavirin, may be used in combination or alternation with the non-immunosuppressive cyclosporins described in this specification. Further non-limiting examples include:

(1) Protease Inhibitors

Examples include substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al, Preparation and use of amino acid derivatives as anti-viral agents, German Patent Pub. DE 19914474; Tung et al. Inhibitors of serine proteases, particularly hepatitis C virus NS3 protease; PCT WO 98/17679), including alphaketoamides and hydrazinoureas, and inhibitors that terminate in an electrophile such as a boronic acid or phosphonate (Llinas-Brunet et al. Hepatitis C inhibitor peptide analogues, PCT WO 99/07734) are being investigated.

Non-substrate-based NS3 protease inhibitors such as 2,4,6-trihydroxy-3-nitro-benzamide derivatives (Sudo K. et al., Biochemical and Biophysical Research Communications, 1997, 238 643 7; Sudo K. et al. Antiviral Chemistry and Chemotherapy, 1998, 9, 186), including RD34082 and RD3-4078, the former substituted on the amide with a 14 carbon chain and the latter processing a para-phenoxyphenyl group are also being investigated.

Sch 68631, a phenanthrenequinone, is an HCV protease inhibitor (Chu M et al., Tetrahedron Letters 37:7229-7232, 1996). In another example by the same authors, Sch 351633, isolated from the fungus Penicillium grieofulvum, was identified as a protease inhibitor (Chu M. et al., Bioorganic and Medicinal Chemistry Letters 9:1949-1952). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, ∀-chymotrypsin, chymase and subtilisin. Qasim M. A. et al., Biochemistry 36:1598-1607, 1997.

U.S. patents disclosing protease inhibitors for the treatment of HCV include, for example, U.S. Pat. No. 6,004,933 to Spruce et al. which discloses a class of cysteine protease inhibitors for inhibiting HCV endopeptidase 2; U.S. Pat. No. 5,990,276 to Zhang et al. which discloses synthetic inhibitors of hepatitis C virus NS3 protease; U.S. Pat. No. 5,538,865 to Reyes et al. Peptides as NS3 serine protease inhibitors of HCV are disclosed in WO 02/008251 to Corvas International, Inc., and WO 02/08187 and WO 02/008256 to Schering Corporation. HCV inhibitor tripeptides are disclosed in U.S. Pat. Nos. 6,534,523, 6,410,531 and 6,420,380 to Boehringer Ingelheim and WO 02/060926 to Bristol Myers Squibb. Diaryl peptides as NS3 serine protease inhibitors of HCV are disclosed in WO 02/48172 to Schering Corporation. Imidazoleidinones as NS3 serine protease inhibitors of HCV are disclosed in WO 02/18198 to Schering Corporation and WO 02/48157 to Bristol Myers Squibb. WO 98/17679 to Vertex Pharmaceuticals and WO 02/48116 to Bristol Myers Squibb also disclose HCV protease inhibitors.

(2) Thiazolidine derivatives which show relevant inhibition in a reverse-phase HPLC assay with an NS3/4A fusion protein and NS5A15B substrate (Sudo K. et al., Antiviral Research, 1996, 32, 9-18), especially compound RD-1-6250, possessing a fused cinnamoyl moiety substituted with a long alkyl chain, RD4 6205 and RD 6193;

(3) Thiazolidines and benzanilides identified in Kakiuchi N. et al. J. FEBS Letters 421, 217-220; Takeshita N. et al. Analytical Biochemistry, 1997, 247, 242-246;

(4) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griseofulvum, which demonstrates activity in a scintillation proximity assay (Chu M. et al, Bioorganic and Medicinal Chemistry Letters 9, 1949-1952);

(5) Helicase inhibitors (Diana G. D. et al., Compounds, compositions and methods for treatment of hepatitis C, U.S. Pat. No. 5,633,388; Diana G. D. et al., Piperidine derivatives, pharmaceutical compositions thereof and their use in the treatment of hepatitis C, PCT WO 97/36554);

(6) Nucleotide polymerase inhibitors and gliotoxin (Ferrari R. et al. Journal of Virology, 1999, 73, 1649-1654), and the natural product cerulenin (Lohmann V. et al. Virology, 1998, 249, 108-118);

(7) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 199,181, 251-257); or an antisense sequence complementary to any part of the HCV genome which increases effectiveness of therapy.

(8) Inhibitors of IRES-dependent translation (Ikeda N et al., Agent for the prevention and treatment of hepatitis C, Japanese Patent Pub. JP-08268890; Kai Y et al. Prevention and treatment of viral diseases, Japanese Patent Pub. JP-10101591);

(9) Ribozymes, such as nuclease-resistant ribozymes (Maccjak, D. J. et al., Hepatology 1999, 30, abstract 995) and those directed in U.S. Pat. No. 6,043,077 to Barber et al., and U.S. Pat. Nos. 5,869,253 and 5,610,054 to Draper et al.; and

(10) Nucleoside analogs have also been developed for the treatment of Flaviviridae infections. See e.g., patent application WO 2004/002422 A2 entitled: “2′-C-Methyl-3′-O-L-Valine Ester Ribofuranosyl Cytidine For Treatment of Flaviviridae Infections”, and U.S. Pat. No. 6,812,219.

Idenix Pharmaceuticals discloses the use of branched nucleosides in the treatment of flaviviruses (including HCV) and pestiviruses in International Publication Nos. WO 01/90121 and WO 01/92282. Specifically, a method for the treatment of hepatitis C infection (and flaviviruses and pestiviruses) in humans and other host animals is disclosed in the Idenix publications that includes administering an effective amount of a biologically active 1′, 2′, 3′ or 4′-branched B-D or B-L nucleosides or a pharmaceutically acceptable salt or prodrug thereof, administered either alone or in combination with another antiviral agent, optionally in a pharmaceutically acceptable carrier.

Other patent applications disclosing the use of certain nucleoside analogs to treat hepatitis C virus include: PCTCA00/01316 (WO 01/32153; filed Nov. 3, 2000) and PCT/CA01/00197 (WO 01/60315; filed Feb. 19, 2001) filed by BioChem Pharma, Inc., (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication Nos. WO 01/79246 (filed Apr. 13, 2001), WO 02/32920 (filed Oct. 18, 2001) and WO 02/48165 by Pharmasset, Ltd.

PCT Publication No. WO 99/43691 to Emory University, entitled “2′-Fluoronucleosides” discloses the use of certain 2′-fluoronucleosides to treat HCV.

Eldrup et al. (Oral Session V, Hepatitis C Virus, Flaviviridae; 16^{th} International Conference on Antiviral Research (Apr. 27, 2003, Savannah, Ga.)) described the structure activity relationship of 2′-modified nucleosides for inhibition of HCV.

Bhat et al. (Oral Session V, Hepatitis C Virus, Flaviviridae, 2003 (Oral Session V, Hepatitis C Virus, Flaviviridae; 16^{th} International conference on Antiviral Research (Apr. 27, 2003, Savannah, Ga.); p A75) describes the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.)p A76) also described the effects of the 2′-modified nucleosides on HCV RNA replication.

11. Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antitoxidants (U.S. Pat. No. 5,922,757 to Chojkier et al.), squalent, amantadine, bile acids (U.S. Pat. No. 5,846,99964 to Ozeki et al.), N-(phosphonoacetyl)-L-aspartic acid) U.S. Pat. No. 5,830,905 to Diana et al.), benzenedicarboxamides (U.S. Pat. No. 5,633,388 to Diane et al.), polyadenylic acid derivatives (U.S. Pat. No. 5,496,546 to Wang et al.), 2′3′-dideoxyinosine (U.S. Pat. No. 5,026,687 to Yarchoan et al.), benzimidazoles (U.S. Pat. No. 5,891,874 to Colacino et al.), plant extracts (U.S. Pat. No. 5,837,257 to Tsai et al., U.S. Pat. No. 5,725,859 to Omer et al., and U.S. Pat. No. 6,056,961) and piperidines (U.S. Pat. No. 5,830,905 to Diana et al.).

12. Receptor agonists such as such as isatoribine (ANA975 and ANA245) a nucleoside analog which is a TOLL-like receptor agonist (U.S. Pat. Nos. 5,041,426 and 4,880,784)

13. Substituted thioaryl urea derivatives such as 1-(4-pentyloxy-3-trifluoromethylphenyl)-3-(pyridine-3-carbonyl)thiourea, (U.S. Publication 2004/0138205, U.S. Ser. No. 10/716,175).

14. Compounds which increase effectiveness of combination therapy including an antifolate, a 5-fluoropyrimidine (including 5-fluorouracil), a cytidine analogue such as β-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine, an alkylating agent (including but not limited to busulfan, chlorambucil, cyclophosphamide, ifofamide, mechlorethamine, melphalan, and thiotepa), nonclassical alkylating agents, platinum containing compounds, bleomycin, an anti-tumor antibiotic, an anthracycline such as doxorubicin and dannomycin, an anthracenedione, topoisomerase II inhibitors, hormonal agents (including but not limited to corticosteroids (dexamethasone, prednisone, and methylprednisone), androgens such as fluoxymesterone and methyltestosterone, estrogens such as diethylstilbesterol, antiestrogens such as tamoxifen, LHRH analogues such as leuprolide, antiandrogens such as flutamide, aminoglutethimide, megestrol acetate, and medroxyprogesterone), asparaginase, carmustine, lomustine, hexamethyl-melamine, dacarbazine, mitotane, streptozocin, cisplatin, carboplatin, levamasole, and leucovorin. The compounds of the present invention can also be used in combination with enzyme therapy agents and immune system modulators such as an interferon, interleukin, tumor necrosis factor, macrophage colony-stimulating factor and colony stimulating factor.

We claim:

1. Use of a cyclosporin in the preparation of a pharmaceutical composition for preventing or treating Flaviviridae infections or Flaviviridae induced disorders wherein the cyclosporin (i) binds to human recombinant cyclophilin with a binding ratio (BR) of less than 0.7, BR being the log to the base 10 of the ratio of the IC₅₀ of the cyclosporin to the IC₅₀ in a simultaneous test of cyclosporin A as measured in a competitive ELISA test; and (ii) has an activity in the Mixed lymphocyte Reaction of not more than 5% that of cyclosporin A.

2. Use of a cyclosporin according to claim 1 in the preparation of a pharmaceutical composition for inhibiting Flaviviridae virus replication.

3. Use of a cyclosporin according to claim 2 wherein the Flaviviridae virus is a flavivirus, pestivirus, or hepacivirus.

4. Use according to claim 1, wherein the cyclosporin is a compound of Formula I 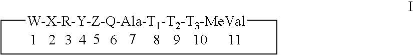
wherein
W is MeBmt, dihydro-MeBmt, 8′-hydroxy-MeBmt or O-acetyl-MeBmt¹;
X is αAbu, Val, Thr, Nva or 0-methyl threonine (MeOThr);
R is Pro, Sar, (D)-MeSer, (D)-MeAla, or (D)-MeSer(Oacetyl);
Y is MeLeu, thioMeLeu, γ-hydroxy-MeLeu, MeIle, MeVal, MeThr, MeAla, MeaIle or MeaThr; N-ethylVal, N-ethylIle, N-ethylThr, N-ethylPhe, N-ethylTyr or N-ethylThr(Oacetyl)
Z is Val, Leu, MeVal or MeLeu,
Q is MeLeu, γ-hydroxy-MeLeu, MeAla or Pro,
T₁ is (D)Ala or Lys,
T₂ is MeLeu or γ-hydroxy-MeLeu, and
T₃ is MeLeu or MeAla;
a compound of Formula Ia 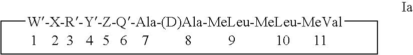
in which W′ is MeBmt, dihydro-MeBmt or 8′-hydroxy-MeBmt;
X is αAbu, Val, Thr, Nva or 0-methyl threonine (MeOThr);
R′ is Sar, (D)-MeSer, (D)-MeAla, or (D)-MeSer(Oacetyl);
Y′ is MeLeu, γ-hydroxy-MeLeu, MeIle, MeVal, MeThr, MeAla, MeaIle or MeaThr; N-ethylVal, N-ethylIle, N-ethylThr, N-ethylPhe, N-ethylTyr or N-ethylThr(Oacetyl)
Z is Val, Leu, MeVal or MeLeu; and
Q′ is MeLeu, γ-hydroxy-MeLeu or MeAla.
or a compound of formula II 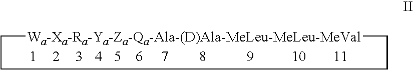
wherein
Wₐ is 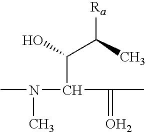
wherein Rₐ is a residue of formula Ic or Id
—CH₂—CH═CH—CH₂—R₄ Ic or —CH₂—SH—R′₄ Id
in which R₄ is C₁₋₄alkylthio, aminoC₁₋₄alkylthio, C₁₋₄alkylaminoC₁₋₄alkylthio, diC₁₋₄alkylamino-C₁₋₄alkylthio, pyrimidinylthio, thiazolylthio, N—C₁₋₄alkylimidazolylthio, hydroxyC₁₋₄alkylphenylthio, hydroxyC₁₋₄alkylphenoxy, nitrophenylamino or 2-oxopyrmidin-1-yl, and R′₄ is C₁₋₄alkyl,
Xₐ is Abu;
Rₐ is —NMe-CH(R_{b})—CO— wherein R_{b} is H or —S-Alk-R₀ in which Alk-R₀ is methyl; or Alk is straight or branched C₂₋₆alkylene or C₃₋₆cycloalkylene and R₀ is H; OH; COOH; C₂₋₅alkoxy-carbonyl; NR₁R₂ in which each of R₁ and R₂, independently, is selected from H, C₁₋₄alkyl, C₂₋₄alkenyl, C₃₋₆cycloalkyl and phenyl each optionally substituted by halogen, C₁₋₄alkoxy, C₂₋₅alkoxycarbonyl, amino, C₁₋₄alkylamino and/or diC₁₋₄alkyl-amino, and benzyl and a heterocyclic radical, said benzyl and heterocyclic radicals being saturated or unsaturated and containing 5 or 6 ring members and 1 to 3 heteroatoms, or R₁ and R₂ form, together with the nitrogen atom to which they are attached, a 4- to 6 membered heterocycle which may contain another heteroatom chosen from nitrogen, oxygen and sulphur, and which is optionally substituted by C₁₋₄alkyl, phenyl or benzyl; or each of R₁ and R₂, independently, is a radical of formula Ib 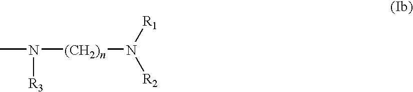
in which R₁ and R₂ are as defined above, R₃ is H or C₁₋₄alkyl and n is an integer ranging from 2 to 4;
Yₐ is MeLeu or γ-hydroxy-MeLeu;
Zₐ is Val; and
Qₐ is MeLeu, with the proviso that R_{b} is not H when Yₐ is MeLeu,
or a pharmaceutically acceptable salt thereof.

5. A pharmaceutical composition for preventing or treating Flaviviridae infections or Flaviviridae induced disorders, comprising a cyclosporin according to claim 1 together with one or more pharmaceutically acceptable diluents or carriers therefor.

6. A pharmaceutical combination comprising a) a first agent which is a cyclosporin according to claim 1, and b) a co-agent having anti-Flaviviridae properties.

7. A pharmaceutical combinaDon for use in the prevention or treatment of Flaviviridae infections or Flaviviridae induced disorders, comprising a) a first agent which is a cyclosporin according to claim 1, and b) a co-agent selected from an agent having anti-Flaviviridae properties, an anti-fibrotic agent, an immune modulating agent or a SIP receptor agonist.

8. The pharmaceutical combination of claim 6 wherein the co-agent having anti-Flaviviridae virus properties is selected from the group consisting of an interferon, ribavirin, interleukin, NS3 protease inhibitor, cystein protease inhibitor, phenanthrenequinone, thiazolidine derivative, thiazolidine, benzanilide, a helicase inhibitor, a polymerase inhibitor, nucleoside analogue, gliotoxin, cerulenin, antisense phosphorothioate oligodeoxynucleotides, inhibitors of IRES-dependent translation, and a ribozyme.

9. A method for preventing or treating Flaviviridae infections or Flaviviridae induced disorders in a subject in need thereof, comprising administering to said subject a therapeutically effective amount of a cyclosporin according to claim 1.

10. A method for inhibing Flaviviridae virus replication in a medium, comprising applying to the medium an effective amount of a cyclosporin according to claim 1.

11. A method for inhibihng Flaviviridae virus replication in a subject in need thereof, comprising administering to the subject a therapeutically effective amount of a cyclospohn according to claim 1.

12. A method according to claim 9, comprising co-administration concomitantly or in sequence of a therapeutically effective amount of a cyclosporin as defined in claim 1 and a co-agent selected from an agent having anti-Flaviviridae properties, an anti-fibrotic agent an immune modulating agent or a S1P receptor agonist

13. The method of claim 12 wherein the co-agent having anti-Flaviviridae properties is selected from the group consisting of an interferon, ribavirin, interleukin, NS3 protease inhibitor, cystein protease inhibitor, phenanthrenequinone, thiazolidine derivative, thiazolidine, benzanilide, a helicase inhibitor, a polymerase inhibitor, nucleoside analogue, gliotoxin, cerulenin, antisense phosphorothioate oligodeoxynucleotides, inhibitors of IRES-dependent translation, and a ribozyme.
